(19) United States
(12) Patent Application Publication
(10) Pub. No.: US 2024/0124664 A1
(43) Pub. Date: Apr. 18, 2024

(54) Hybrid Nanomaterials Comprising Porous Graphene and Palladium Nanoparticles

(71) Applicants: Pilgyu Kang, Annandale, VA (US); Seung Min Lee, Fairfax, VA (US); Daniel Michael Scott, Chantilly, VA (US); Farbod Moghaddam, Sterling, VA (US); Byoung Gak Kim, Seogu (KR); Minsu Kim, Seochu-Gu (KR)

(72) Inventors: Pilgyu Kang, Annandale, VA (US); Seung Min Lee, Fairfax, VA (US); Daniel Michael Scott, Chantilly, VA (US); Farbod Moghaddam, Sterling, VA (US); Byoung Gak Kim, Seogu (KR); Minsu Kim, Seochu-Gu (KR)

(21) Appl. No.: 18/373,847

(22) Filed: Sep. 27, 2023

Related U.S. Application Data
(60) Provisional application No. 63/411,650, filed on Sep. 30, 2022.

(51) Int. Cl. C08J 5/18; C08K 3/08; C08K 7/24; G01N 33/00

(52) U.S. Cl. CPC C08J 5/18; C08K 3/08; C08K 7/24; G01N 33/005; C08J 2371/00; C08K 2201/003; C08K 2201/011; C08K 2201/014

(57) ABSTRACT

Hybrid nanomaterials, methods of making the nanomaterials, and methods of using the nanomaterials to detect hydrogen gas.

### CROSS-REFERENCE TO RELATED APPLICATIONS

This application claims priority to U.S. Provisional Application No. 63/411,650, filed Sep. 30, 2022, the entirety of which is incorporated into this application by reference.

### BACKGROUND

Hydrogen has been extensively investigated as a renewable energy source owing to its abundance in nature, high energy content, and clean combustion reaction with byproducts of only water and heat. To realize hydrogen as an energy carrier, effective hydrogen gas leakage detection systems for hydrogen storing or generation are important because of potential explosion risks. Carbon nanomaterials including carbon nanotubes, graphene, and carbon nanofibers have been explored as functional materials for the electrochemical detection of hydrogen gas. The superior mechanical and chemical stability, high carrier mobility, and large specific surface areas of such materials are suitable for application to highly sensitive and wearable hydrogen detection systems.

### SUMMARY

In one aspect, the method of making a hybrid nanomaterial comprises: (a) providing a solution comprising a polymer having intrinsic microporosity and a palladium(II) ligand in an organic solvent, wherein the polymer and the ligand are least partially soluble in the organic solvent; (b) removing the organic solvent to provide a film comprising the polymer and the ligand; and (c) irradiating the film with an infrared laser to form the hybrid nanomaterial.

The hybrid nanomaterial can be used to detect hydrogen gas. In one aspect, the method of detecting hydrogen gas comprises exposing the hybrid nanomaterial to an environment in which hydrogen gas is to be detected and determining any increase in resistance exhibited by the hybrid nanomaterial while exposed to the environment, thereby indicating the presence or absence of hydrogen gas.

In one aspect, the hybrid nanomaterial comprises porous graphene having an x-ray diffraction pattern containing peaks at about 43° and 26° 2θ; and palladium nanoparticles dispersed in the porous graphene, the palladium nanoparticles having an x-ray diffraction pattern containing peaks at about 39°, 45°, 66°, and 81° 2θ.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1 is a schematic illustration of a wireless and real-time H₂ sensing system composed of the laser-induced nanohybrid sensors and wireless signal transmission module.

FIG. 2 is a schematic illustration of the fabrication procedures for the nanohybrid structures. The procedure consists of a laser photothermochemical single-step method for synchronous nanoassembly of palladium nanoparticles (PdNPs) and 3D porous graphene structures. The homogeneously mixed polymer film of polymers of intrinsic microporosity-1 (PIM-1) and palladium ligands is laser-photothermally treated and the nanohybrid is formed on the surface of the film via photothermal and photochemical processes.

FIG. 3A is a photograph showing the homogeneous Pd-ligand/PIM-1 film with 30 wt. % of Pd-ligand.

FIG. 3B is a photograph showing the patterned nanoassembly arrays in sizes from milli- to micro-meter scale on the homogeneous substrate with 0 wt. %, 10 wt. %, 20 wt. %, and 30 wt. % of Pd-ligand (scale bars: 1 mm).

FIG. 4A is a scanning electron microscope (SEM) image showing cross-section of hybrid structures generated from 0 wt. % Pd-ligands and PIM-1 homogenous films.

FIG. 4B is a scanning electron microscope (SEM) image showing cross-section of hybrid structures generated from 10 wt. % Pd-ligands and PIM-1 homogenous films.

FIG. 4C is a scanning electron microscope (SEM) image showing cross-section of hybrid structures generated from 20 wt. % Pd-ligands and PIM-1 homogenous films.

FIG. 4D is a scanning electron microscope (SEM) image showing cross-section of hybrid structures generated from 30 wt. % Pd-ligands and PIM-1 homogenous films.

FIG. 4E is a high-resolution tunneling electron microscope (HR-TEM) image showing hybrid structures generated from 0 wt. % Pd-ligand and PIM-1 homogenous films. The inset shows the lattice structure of porous graphene.

FIG. 4F is a high-resolution tunneling electron microscope (HR-TEM) image showing hybrid structures generated from 10 wt. % Pd-ligand and PIM-1 homogenous films. The inset shows the lattice structure of Pd NPs.

FIG. 4G is a high-resolution tunneling electron microscope (HR-TEM) image showing hybrid structures generated from 20 wt. % Pd-ligand and PIM-1 homogenous films. The inset shows the lattice structure of Pd NPs.

FIG. 4H is a high-resolution tunneling electron microscope (HR-TEM) image showing hybrid structures generated from 30 wt. % Pd-ligand and PIM-1 homogenous films. The inset shows the lattice structure of Pd NPs.

FIG. 5A is a scanning electron microscope (SEM) image showing cross-section and surface of nanoassembly generated from 0 wt. % Pd-ligand and PIM-1 homogenous films.

FIG. 5B is a scanning electron microscope (SEM) image showing cross-section and surface of nanoassembly generated from 10 wt. % Pd-ligand and PIM-1 homogenous films.

FIG. 5C is a scanning electron microscope (SEM) image showing cross-section and surface of nanoassembly generated from 20 wt. % Pd-ligand and PIM-1 homogenous films.

FIG. 5D is a scanning electron microscope (SEM) image showing cross-section and surface of nanoassembly generated from 30 wt. % Pd-ligand and PIM-1 homogenous films.

FIG. 6A is a scanning electron microscope (SEM) image showing cross-section at position A of nanoassembly generated from 30 wt. % Pd-ligand and PIM-1 homogenous films.

FIG. 6B is a scanning electron microscope (SEM) image showing cross-section at position B of nanoassembly generated from 30 wt. % Pd-ligand and PIM-1 homogenous films.

FIG. 6C is a scanning electron microscope (SEM) image showing cross-section at position C of nanoassembly generated from 30 wt. % Pd-ligand and PIM-1 homogenous films.

FIG. 7 is a scatter plot showing Ar adsorption-desorption isotherms: the Brunauer-Emmett-Teller (BET) specific surface area of the laser-induced porous graphene from PIM-1 was determined to be 162.2 m²·g⁻¹.

FIG. 8A is X-ray diffraction (XRD) spectra of the hybrid structures.

FIG. 8B is Raman spectra of the hybrid structures.

FIG. 8C is X-ray photoelectron spectroscopy (XPS) of the hybrid structures in the C1s region.

FIG. 8D is X-ray photoelectron spectroscopy (XPS) of the hybrid structures in the Pd3d region.

FIG. 9 is X-ray diffraction (XRD) spectra of the polymer of intrinsic microporosity-1 (PIM-1) and laser-induced porous graphene generated from PIM-1.

FIG. 10 is X-ray photoelectron spectroscopy (XPS) spectra and chemical compositions of the hybrid structures generated from (a) 0 wt. %, (b) 10 wt. %, (c) 20 wt. %, and (d) 30 wt. % Pd-ligand and PIM-1 homogeneous films

FIG. 11A is a plot showing hydrogen detection sensitivity (ΔRₘₐₓ/Rₒ) and response time (τ) of hydrogen sensing devices based on the nanohybrid with various Pd concentrations (0, 10, 20, 30 wt. %) at 10 ppm of H₂.

FIG. 11B is a plot showing sensitivity (ΔRₘₐₓ/Rₒ) and the response time (τ) of the nanohybrid sensor device with optimal Pd-30 wt. % for various H₂ concentrations (1, 5, 10, 20, 50 ppm).

FIG. 11C is a plot showing cyclic hydrogen sensing with the nanohybrid sensor device (Pd-30 wt. %) for various hydrogen concentrations of 1, 5, and 50 ppm). Mechanical flexibility and sensing reliability test of the nanohybrid sensor device at 10 ppm H₂ sensing under

FIG. 11D is a plot showing mechanical flexibility and sensing reliability test of the nanohybrid sensor device at 10 ppm H₂ sensing under bending strain (ε) up to 0.22%.

FIG. 11E is a plot showing mechanical flexibility and sensing reliability test of the nanohybrid sensor device at 10 ppm H₂ sensing under cyclic bending (ε=0.22%) up to 10,000 cycles.

FIG. 11F is a plot showing mechanical flexibility and sensing reliability test of the nanohybrid sensor device at 10 ppm H₂ sensing under cyclic twisting of ±90° up to 10,000 cycles.

FIG. 12 are current-voltage characteristic curves of the laser-induced nanoassembly generated from PIM-1/Pd acetate homogeneous films with various Pd precursor concentrations (0, 10, 20, 30 wt %).

FIG. 13 is a linear regression showing the sensitivity of the laser-induced nanoassembly hydrogen sensor devices generated from PIM-1/Pd acetate homogeneous films with various Pd concentrations (0, 10, 20, 30 wt %).

FIG. 14 is a plot showing the dynamic responses of the laser-induced nanoassembly based H₂ gas sensor generated from plain PIM-1 and PIM-1/Pd acetate 30 wt. % films for 10 ppm of H₂.

FIG. 15 is a plot and a schematic that shows wireless, real-time hydrogen sensing with the integrated detection system in emulating H₂ leakage of various hydrogen concentration (1, 5, 10, 20, 50 ppm). The data of ΔR/Rₒ is monitored on the developed application of a mobile phone at the distance of 20 m away from the sensor device (Inset: Consistent sensitivity of the integrated detection system for 1 ppm of H₂ leakage at various distances (0, 5, 10, 15, and 20 m))

FIG. 16 is a plot that shows the dynamic responses of Bluetooth wireless sensing device at various hydrogen concentrations (1, 5, 10, 20, 50 ppm).

### DETAILED DESCRIPTION

In one aspect, disclosed is a hybrid nanomaterial comprising porous graphene and palladium nanoparticles dispersed in the porous graphene. In one aspect, the hybrid nanomaterial comprises porous graphene having an x-ray diffraction pattern containing peaks at about 43° and about 26° 2θ; and palladium nanoparticles dispersed in the porous graphene, the palladium nanoparticles having an x-ray diffraction pattern containing peaks at about 39°, about 45°, about 66°, and about 81° 2θ. When the term “about” precedes an x-ray diffraction peak, the stated value can vary by ±1° 2θ. In a further aspect, the porous graphene has an x-ray diffraction pattern containing peaks at 42.8° and 25.9° 2θ, and the palladium nanoparticles have an x-ray diffraction pattern containing peaks at 39.6°, 45.5°, 66.5°, and 81.5° 2θ. The palladium nanoparticle peaks generally correspond to the (111), (200), (220), and (311) plane reflections of the nanoparticles. The x-ray diffraction pattern of the porous graphene generally indicates that the graphene is oriented in sheets in the dominant (111) plane and stacked in the (002) plane.

In one aspect, the porous graphene has BET specific surface area ranging from about 150 m²/g to about 180 m²/g. In a further aspect, the porous graphene has a BET specific surface area of about 162.2 m²/g.

When the term “about” precedes a numerical value other than an x-ray diffraction peak, the numerical value can vary within ±10% unless specified otherwise.

In one aspect, the average diameter of the palladium nanoparticles ranges from about 5 nm to about 12 nm as determined by transmission electron microscopy. In a further aspect, the average diameter of the palladium nanoparticles ranges from about 6 nm to about 10 nm. Specific non-limiting but measured examples of the palladium nanoparticle diameters include 8.13±2.76, 8.63±1.83, and 8.91±1.58 nm. In a further aspect, the statistical mean diameter of the palladium nanoparticles is 7.06±2.08.

In one aspect, the hybrid nanomaterial has a Raman spectrum containing peaks at about 1,350 cm⁻¹, about 1580 cm⁻¹, and about 2690 cm⁻¹. These peaks generally correspond wo the D, G, and 2D peaks, respectively, indicating the presence of randomly stacked sp 2-hybridized graphene layers in the porous 3D structure of the hybrid nanomaterial.

In one aspect, the porous graphene has a lattice fringe spacing ranging from about 0.2 nm to about 0.5 nm as determined by transmission electron microscopy. In a further aspect, the porous graphene has a statistical mean lattice fringe spacing of about 0.34 nm as determined by transmission electron microscopy. In a further aspect, the porous graphene has an average pore diameter of 2 nm or less as determined by transmission electron microscopy.

In one aspect, the palladium nanoparticles have an atomic lattice spacing ranging from about 0.2 to about 0.3 nm as determined by transmission electron microscopy. In a further aspect, the palladium nanoparticles have an atomic lattice spacing of about 0.23 nm as determined by transmission electron microscopy.

In one aspect, the hybrid nanomaterial comprises from about 1% to about 5% palladium nanoparticles by weight of the porous graphene. In a further aspect, the hybrid nanomaterial comprises from about 2% to about 4% palladium nanoparticles by weight of the porous graphene. In a specific aspect, the hybrid nanomaterial comprises up to 3.3% palladium nanoparticles by weight of the porous graphene.

The hybrid nanomaterials may be made by a fast, photothermochemical processing method, using a laser (e.g. an infrared laser) and polymer films synthesized from carbon and palladium precursors. In one aspect, the method for manufacturing the hybrid nanomaterials may include one or more of: (a) providing a solution comprising a polymer having intrinsic microporosity and a palladium(II) ligand in an organic solvent, wherein the polymer and the ligand are at least partially soluble in the organic solvent; (b) removing the organic solvent to provide a film comprising the polymer and the ligand; and (c) irradiating the film with laser light, thereby forming the hybrid nanomaterial. The laser light may be infrared light. In an exemplary embodiment the laser light is generated by a CO₂ infrared laser having a wavelength (λ) of 10.6 μm. The polymer film can in some aspects be formed by solution casting methods, followed by drying or another suitable method to remove the volatile organic solvent. In one aspect, the polymer and the ligand are soluble in the organic solvent.

In one aspect, the polymer used has the following repeating structure:

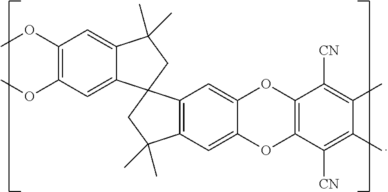

In one aspect, the palladium(II) ligand is palladium(II) acetate (Pd²⁺(OAc)₂). In a further aspect, prior to irradiating the film comprises 10-30% by weight of the palladium(II) ligand.

In one aspect, irradiating is performed at 12 Watts, 1,000 laser pulses per inch (PPI), and 50 inches per second. In a further aspect, the organic solvent is chloroform. Also described is a hybrid nanomaterial made by the method.

The hybrid nanomaterials can be used in a variety of applications, including hydrogen gas detection. In one aspect, the method of detecting hydrogen gas comprises exposing the hybrid nanomaterial to an environment in which hydrogen gas is to be detected and determining any increase in resistance exhibited by the hybrid nanomaterial while exposed to the environment, thereby indicating the presence or absence of hydrogen gas. In a further aspect, any increase in resistance is monitored by a computing device in wired or wireless communication with the hybrid nanomaterial. The computing device for example can be a mobile phone or tablet. When in wireless communication with the hybrid nanomaterial, hydrogen can conveniently be detected at a significant distance from the source environment, for example up to 20 meters away. In one aspect, it is contemplated that the hybrid nanomaterial can be integrated into a wearable hydrogen detection system. A hydrogen detection system comprising one or more hybrid nanomaterial sensors is disclosed. The hydrogen hybrid nanomaterial sensor may include one or more hybrid nanomaterials. The hybrid nanomaterial sensors may be configured to change their electrical resistance when exposed to hydrogen gas. The hydrogen detection system may further include a resistance measuring system (e.g. ohmeters, combination of voltmeters and amperemeters) configured to measure the change in resistance caused by the exposure to hydrogen. The value of the change in resistance caused by the exposure to the hydrogen gas may be indicative of the amount of hydrogen absorbed in the nanomaterial. The value of the change in resistance caused by the exposure to the hydrogen gas may be indicative of the amount of hydrogen (e.g. concentration of hydrogen) in the environment. The hydrogen detection sensor may further include communication devices configured to transmit the results of the measurements to an external device such as a computer or a smart phone.

### A. EXAMPLES

The following examples further illustrate this disclosure. The scope of the disclosure and claims is not limited by the scope of the following examples.

### 1. Materials and Experimental

5,5′,6,6′-Tetrahydroxy-3,3,3′,3′-tetramethyl-1,1′-spirobisindane (TTSBI, >97%), and palladium(II) acetate (Pd/Ac, >98%) were purchased from Tokyo Chemical Industry Co., Ltd. Tetrafluoroterephthalonitrile (TFTPN, >98%) was purchased from Matrix Scientific. Potassium carbonate (K2CO3, 99.99%, anhydrous), and chloroform (>99.9%) were purchased from Sigma-Aldrich. Tetrahydrofuran (THF) was purchased from J. T. Baker. Dichloromethane was purchased from Samchun Pure Chemical Co., Ltd. TTSBI and TFTPN were used as monomers. TTSBI was purified by recrystallization from a mixture of dichloromethane and methanol. TFTPN was purified by vacuum sublimation at 150° C. Palladium(II) acetate (>98%) was used as Pd-ligand.

A. Preparation of PIM-1

All glassware was dried in an oven before use. Under N₂ flow, a mixture of TTSBI (10.21 g, 30 mmol), TFTPN (6.00 g, 30 mmol), and anhydrous K₂CO₃ (8.29 g, 60 mmol) was dissolved in DMF (210 mL) in a 500 mL three-necked round-bottomed flask equipped with a condenser. The mixture was magnetically stirred at 55° C. for 72 h. Then, the mixture was cooled at room temperature and 150 mL of THF was added into the flask for removing low molecular weight oligomers. The resulting solution was added into an excess of water and the synthesized polymer was precipitated. The polymer was re-dissolved in THF and the polymer was reprecipitated in methanol (MeOH) to further remove the low molecular weight oligomers. In order to remove remained solvent, the re-precipitated polymer was refluxed in MeOH for 24 h. The yellow PIM-1 was obtained in 75% yield after drying under vacuum for several days. The chemical structure of the PIM-1 was confirmed by 1H NMR (500 MHz, CDCl₃): δH (ppm)=6.81 (2H, s), 6.42 (2H, s), 2.47-1.90 (4H, dd), 1.50-1.15 (12H, br). The molecular weight of PIM-1 was measured using GPC analysis: Mn=54 099, Mw=118 720, PDI=2.19.

B. Fabrication of Porous Graphene and Palladium Nanoparticles Hybrid Structures

PIM-1 (0.5 g) was first dissolved into chloroform (16.7 ml) and 0 wt. %, 10 wt. %, 20 wt. %, and 30 wt. % of palladium acetate were added. The mixture was stirred under ambient condition for 3 h to prepare homogeneous solution. Then, the solution was filtered through 1 μm polytetrafluoroethylene (PTFE) filters, cast into glass dish, and slowly dried at room temperature for 2 days, and the homogeneous films with thickness of ˜100 μm were fabricated.

The 10.6 μm CO₂ laser with 12 W laser power and 1000 laser pulses per inches (PPI) is irradiated on the homogeneous film to produce the nanoassembly. A lasing speed of 50 inches per second was used.

C. Materials Characterizations

¹H nuclear magnetic resonance (¹H NMR) (500 MHz Bruker AVANCE NEO NMR spectrometer using tetramethylsilane (TMS) as the reference with CDCl₃ as solvent) was employed to determine and confirm the chemical structure of fabricated PIM-1. The molecular weight of PIM-1 was confirmed by gel permeation chromatography (GPC, Wyatt Technology) in THF. Scanning electron microscopy (SEM) (Hitachi, SU8230) was utilized to qualitatively investigate the structural morphologies of nanoassembly of 3D porous graphene and palladium nanoparticles. To investigate the detailed morphology and plane structures of the porous graphene and the nanoassembly, transmission electron microscopy (TEM) (TECNAI, G2 T-205) was performed. A Raman spectrometer (Renishaw, inVia Raman Microscope) with a 514 nm laser was used for structural analysis of the 3D porous graphene in nanoassembly. The crystalline structure of the PdNPs was determined by X-ray diffraction (XRD) (Rigaku, Ultima IV). The surface area and micropore size of 3D porous graphene were analyzed with an accelerated surface area and porosimetry system (Micromeritics, ASAP). The X-ray photoelectron spectrometer (Thermo VG Scientific, K-alpha) was utilized to quantitatively investigate the chemical bonding and compositions of PdNPs and 3D porous graphene in the nanoassembly.

D. H₂ Gas Detection Performance of the Nanoassembly Based Sensors

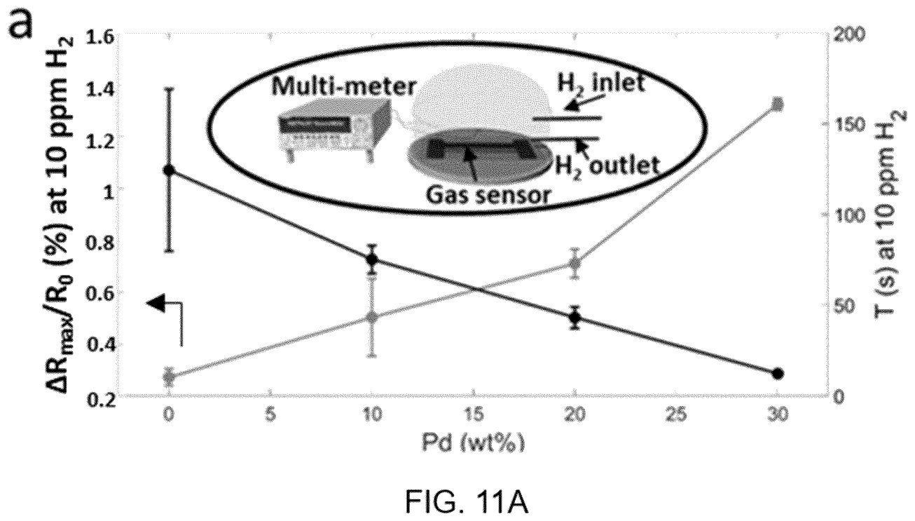

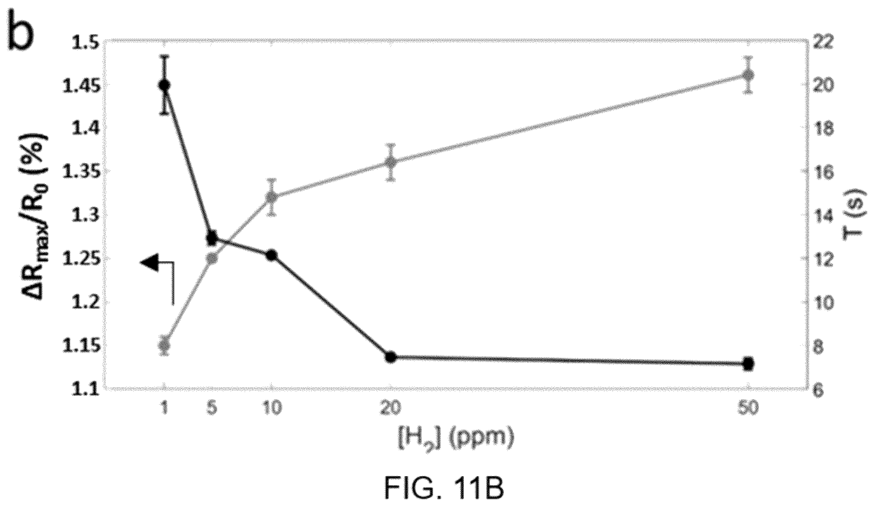

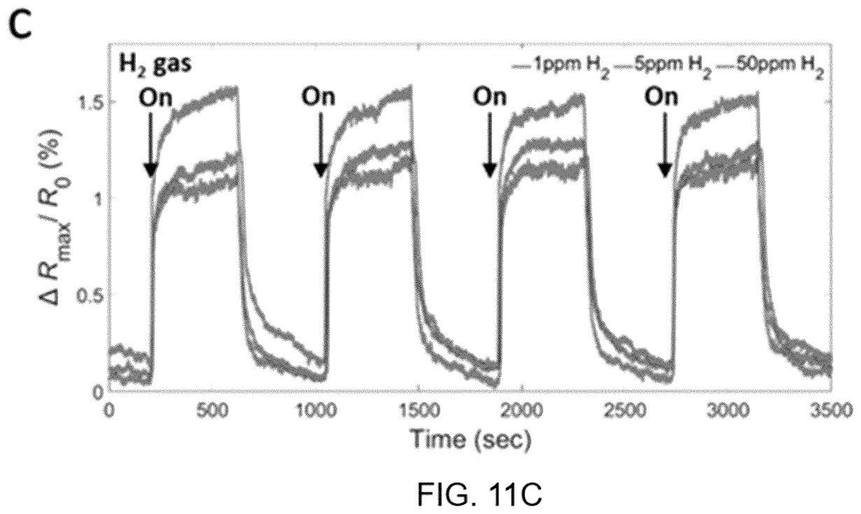

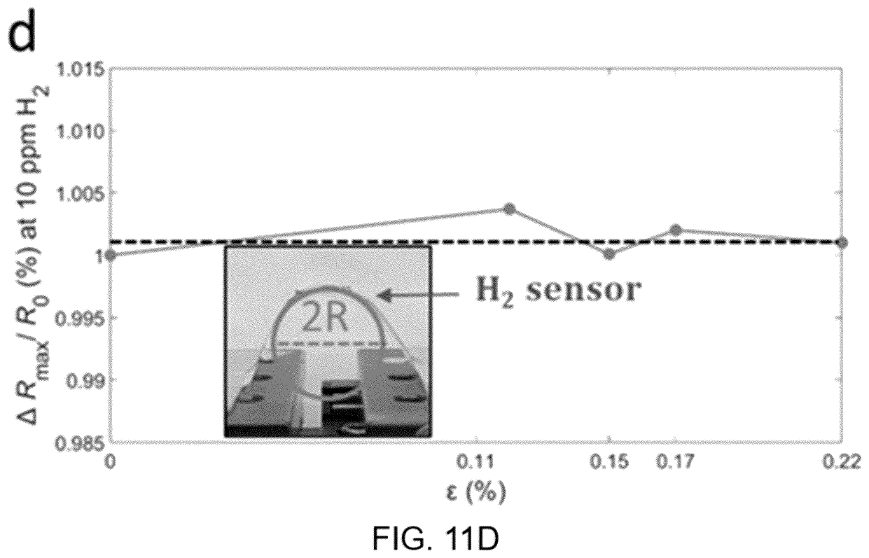

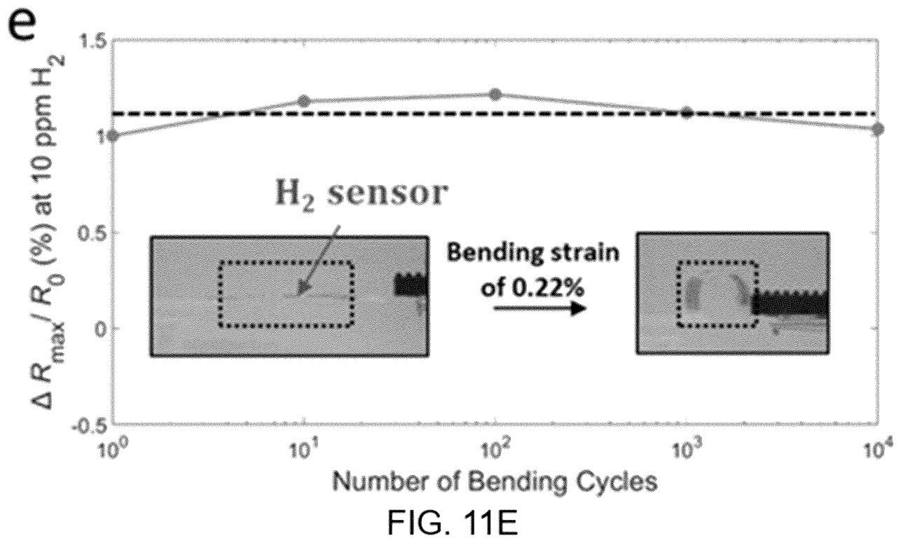

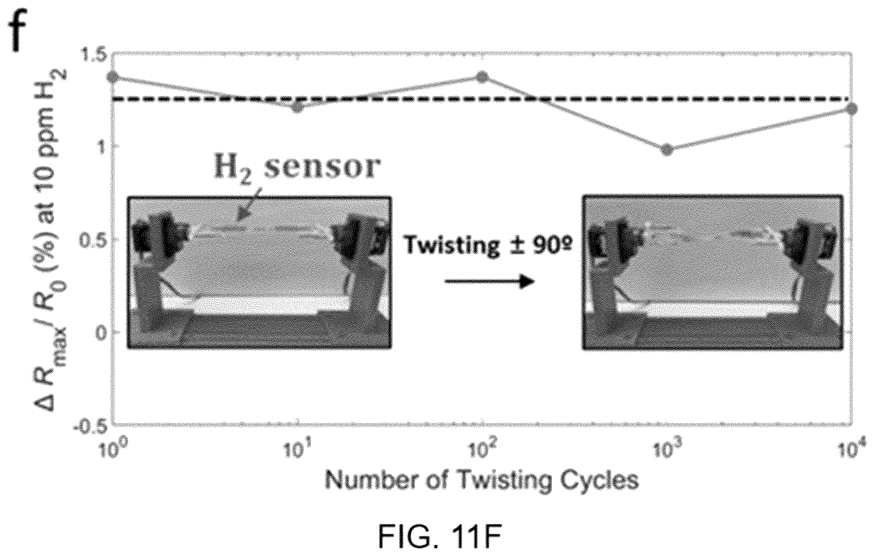

To characterize the H₂ gas detection performance of PdNPs/PIM-1 nanoassembly, the nanoassembly based gas sensors with length of 1 mm and width of 500 μm were fabricated. 100 nm-thick gold electrodes were deposited at both ends of the nanoassembly generated from PIM-1/Pd acetate homogeneous films with four different Pd concentrations (0, 10, 20, 30 wt. %). Then, the gold electrodes were connected with copper wires using conductive silver pastes (1602, Pelco) and dried in a thermal oven at 65° C. for 30 minutes. The detectors were placed inside of the closed chamber and connected to a two-probe-type multi-meter (2614B, Keithley Instruments) as the inset schematics in FIG. 11A. The detectors were vacuum treated for 120 seconds to remove remaining gases in the chamber and contaminants on the nanoassembly. The bias voltage of 0.1 V was applied to induce the current. Various concentrations of H₂ gas (1, 5, 10, 20, 50 ppm) were injected into the chamber by using a mass flow controller (SmartTrak 50, Sierra), injecting controlled amount of H₂ into the chamber with the flow rate of 100 sccm (standard cubic centimeters per minute). The flow rate of H₂ gas was set to 0 sccm, once the injected H₂ gas contents reached to the targeted concentration. All the measurements were performed for 600 s to confirm the hydrogen detector was completely reached the saturation point. The sensitivity of the sensor was measured based on the percent change of resistance from its initial electrical resistance. The response time was evaluated as the time required to reach 36.8% of the maximum resistance of the sensor.

E. Bluetooth Based Wireless Sensing System with Data Collection by Smart Phone

The wireless sensing device was constructed by connecting laser-induced nanoassembly based H₂ gas sensor, Bluetooth (BLE) microcontroller integrated Arduino chip (Adafruit Feather 32u4, Arduino), and a source-meter (2614B, Keithley Instruments) in series. The Arduino was programmed to measure the resistance change in H₂ gas sensor and to transport the measured data into the BLE microcontroller. The mobile application, Bluefruit Connect was downloaded on a smart phone to receive the data from the BLE microcontroller. The H₂ gas detection performance of the nanoassembly based wireless sensor was demonstrated in the same experimental design and methods as those in non-wireless devices. The collected data was received by the smartphone at different distances from the chamber (0, 5, 10, 15, 20 m).

### 2. Example 1: Synthesis of the Graphene and Palladium Nanoparticles Nanoassembly

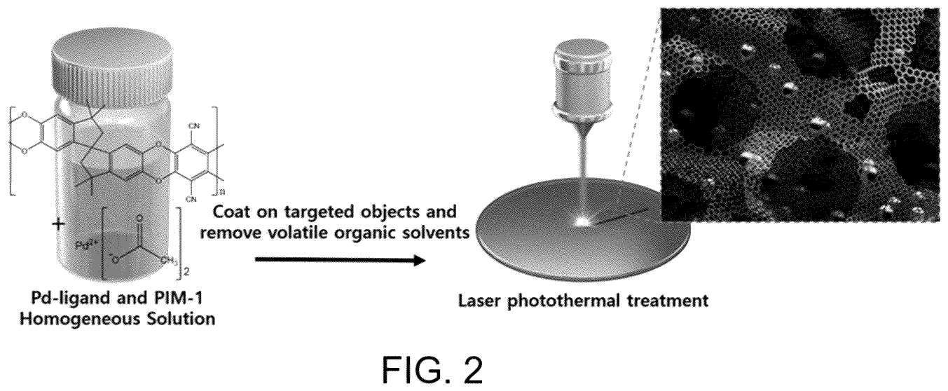

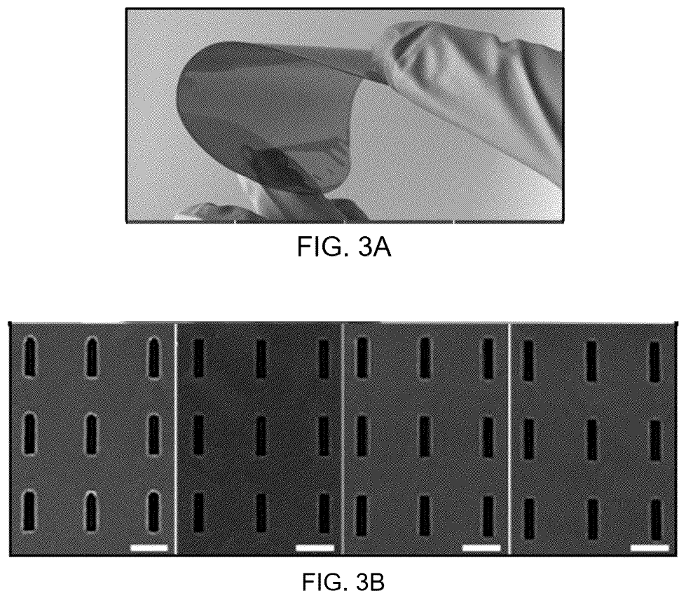

To synthesize the nanohybrid of carbon-encapsulated Pd nanoparticles in porous graphene, the homogeneous polymer films comprising PIM-1 and various amounts of Pd-ligant (0, 10, 20, and 30 wt. %) were prepared via solution casting method. FIG. 3A shows a photograph of the homogeneous Pd-ligand/PIM-1 film with 30 wt. % of Pd-ligand. FIGS. 3A-B are photographs of the patterned nanoassembly arrays in sizes from milli- to micro-meter scale on the homogeneous substrate with 0 wt. %, 10 wt. %, 20 wt. %, and 30 wt. % of Pd-ligand (scale bars: 1 mm). The content of Pd-ligand mixed with PIM-1 in the homogeneous phase was limited to 30 wt. % to obtain mechanically rigid and flexible PIM-1. FIG. 2 is a schematic showing the fabrication procedures for the nanohybrid structures. The CO₂ infrared laser (λ=10.6 μm) was irradiated on the as-fabricated film (laser power, engraving speed, and pulse per inch (PPI) were optimized to 12 W, 50 inch/s, and 1000, respectively) to obtain hybrid nanostructures of PdNP-embedded porous graphene via photothermal effect (or pyrolytic effect). Laser irradiation induced the photothermal excitation of the polymer film through the absorption of photon energy, resulting in a significant increase in temperature within the focused area of the laser, where the carbon atoms in the PIM-1 polymer chains were sp 2-hybridized. The photothermal effect evaporated the volatile by-products from the remained atoms and generated heterogeneous nucleation of vapor bubbles to form micro/meso/macro pore structures.^{[}20] Simultaneously, the organic functional groups in Pd-ligands were decomposed via photon-induced thermal and non-thermal chemical reactions comprising the photophysical mechanism. The combined effects of pyrolysis and photolysis resulted in the breakage of the bonds of Pd ligand molecules, and the Pd atoms nucleated and crystallized to form nanoparticles, generating a nanoassembly with the homogenous dispersion of PdNPs in 3D porous graphene.

### 3. Example 2: Characterization of the Nanoassembly

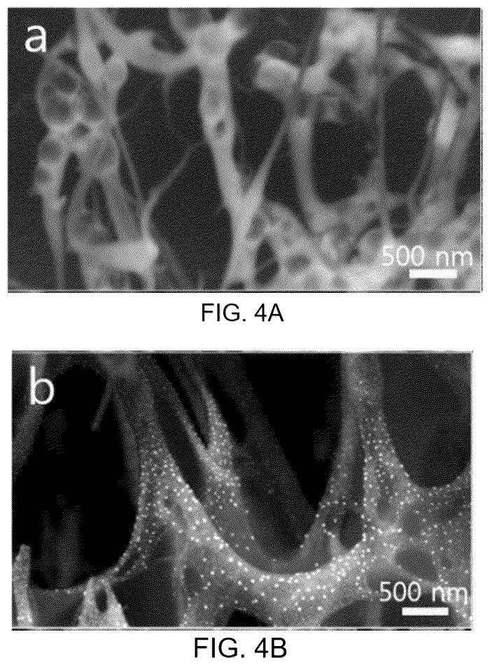

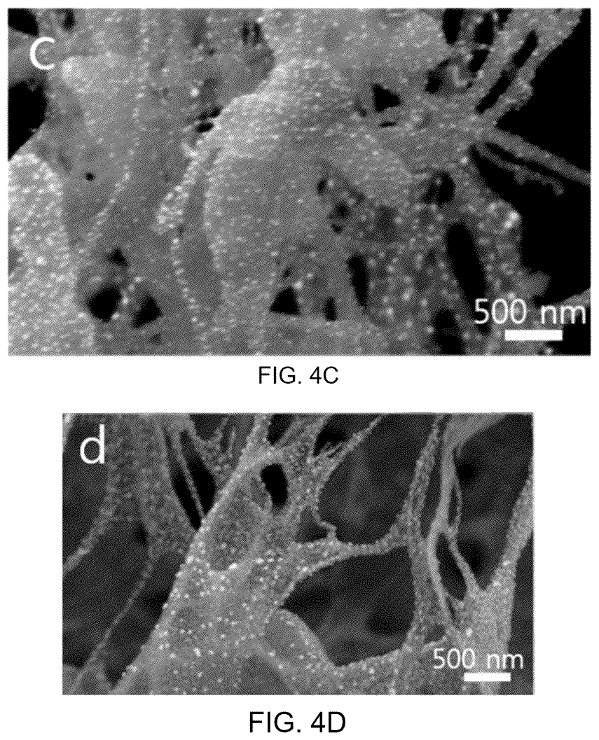

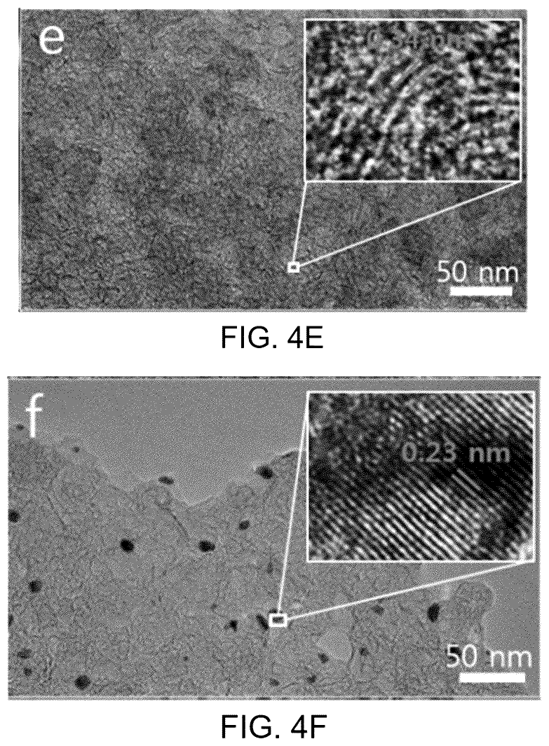

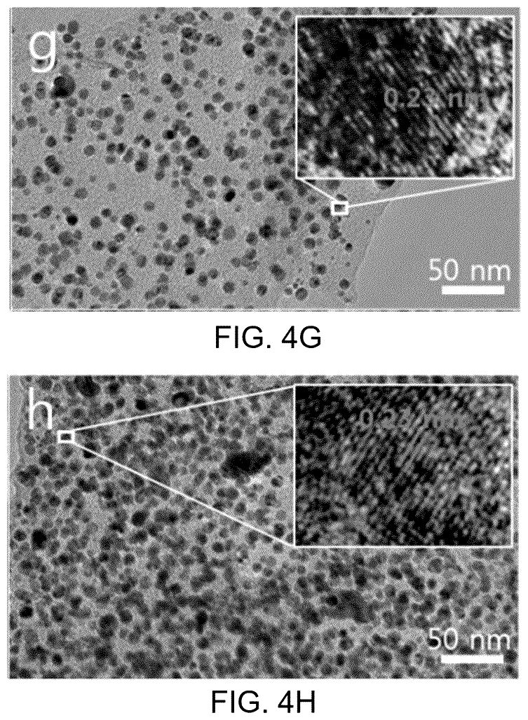

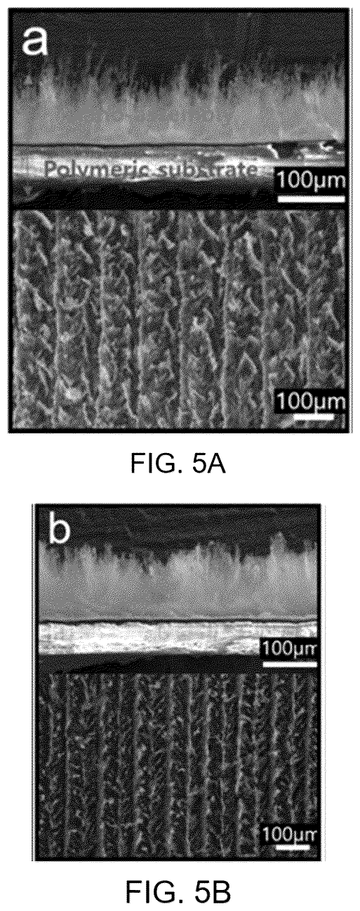

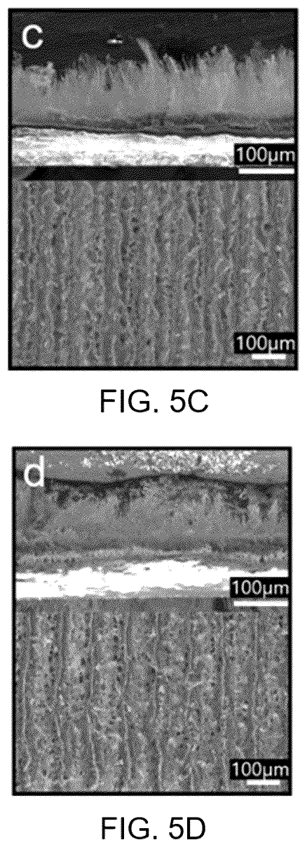

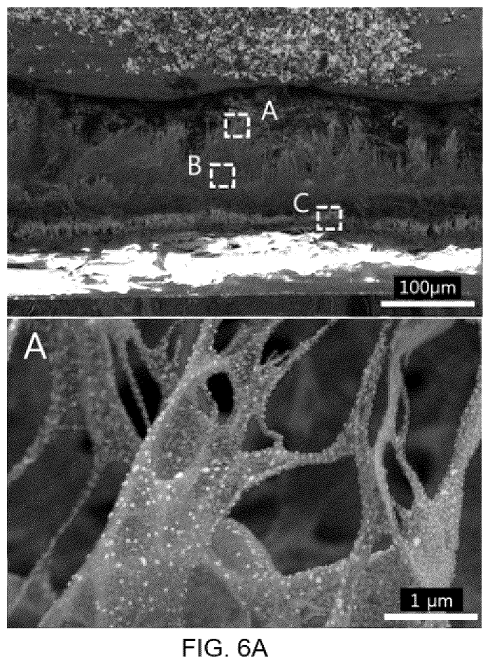

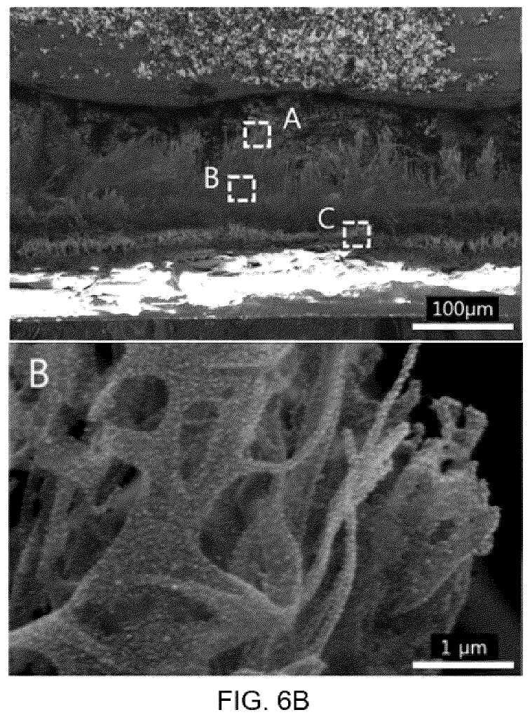

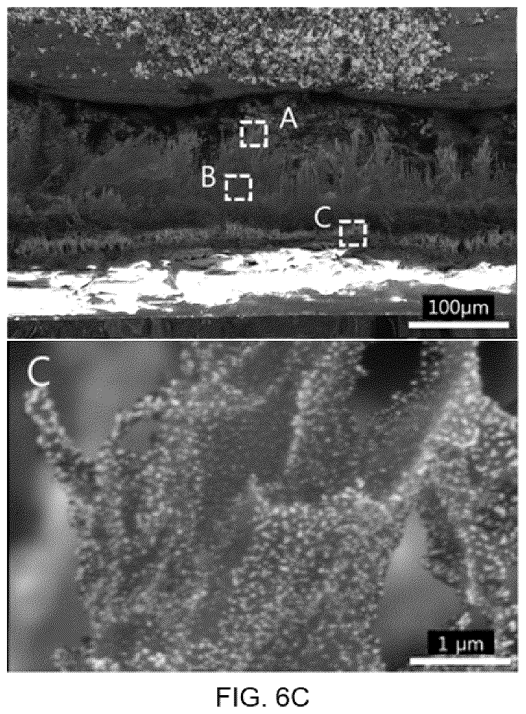

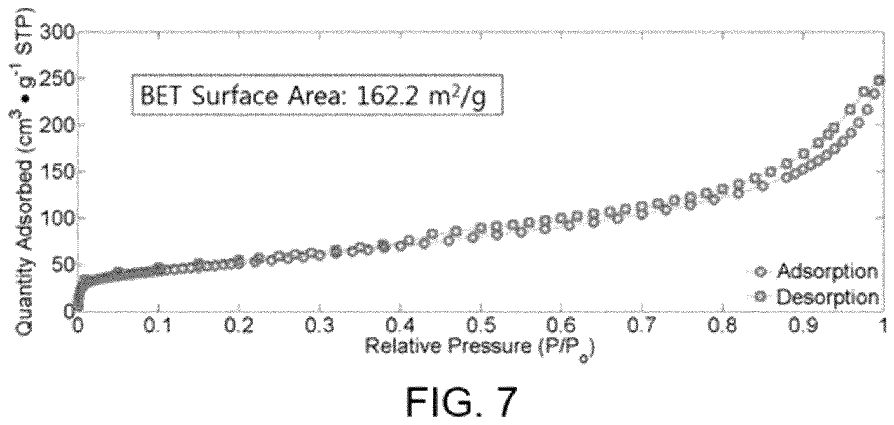

The morphology of the hybrid nanoassembly was examined using scanning electron microscopy (SEM) and high-resolution tunneling electron microscopy (HR-TEM). Images obtained from these techniques are found in FIG. 3. The SEM images are shown in FIG. 4A-H, and they show that Pd nanoparticles were dispersed in porous graphene for all nanohybrids obtained with different contents of the Pd-ligand (0, 10, 20, and 30 wt. %) in PIM-1. As shown in the cross-sectional SEM images in FIG. 5A-D, approximately 130 μm-thick fluttering and porous structured graphene is formed on an approximately 70-μm-thick polymeric substrate. Additional SEM images in FIG. 6A-C show cross-sections at various positions of the nanoassembly generated from 30 wt. % Pd-ligand and PIM-1 homogeneous films and further demonstrate that the laser manufacturing method with mixed polymer precursor films allowed the formation of nanosized Pd particles with uniform NP sizes and distribution in the porous graphene structures with different Pd-ligand contents. Based on the HR-TEM analysis, which is shown in FIG. 3E-H, the average diameter of the PdNPs in the nanohybrids formed using 10, 20, and 30 wt. % Pd-ligand-contained PIM-1 film was estimated to be 8.13±2.76, 8.63±1.83, and 8.91±1.58 nm, respectively. The NP size increased by 6.2% and 9.0% with 20 and 30 wt. % Pd-ligand, respectively, compared to the size of PdNP with 10 wt. % Pd-ligand. The results showed the potential for controlling the NP sizes with variation in the Pd-ligand content. FIG. 3E shows that the spacing of the lattice fringe in porous graphene was 0.34 nm, which corresponded to graphitic carbon stacked on the (002) planes. Furthermore, FIG. 3F-H show that the atomic lattice spacing of PdNPs was estimated to be 0.23 nm for all nanohybrids with varying PdNP compositions, indicating the dominant lattice structure of Pd(111) crystalline planes. The homogeneous nanoparticle structures and dispersions with high crystallinities for various Pd-ligand contents are attributable to the homogeneous mixture of polymer precursors and uniform photon absorption in the polymer films that allow homogenous nucleation and crystallization of Pd atoms. Lastly, FIG. 7 shows Ar adsorption-desorption isotherms. The Brunauer-Emmett-Teller (BET) specific surface area of the laser-induced porous graphene from PIM-1 was determined to be 162.2 m² g⁻¹.

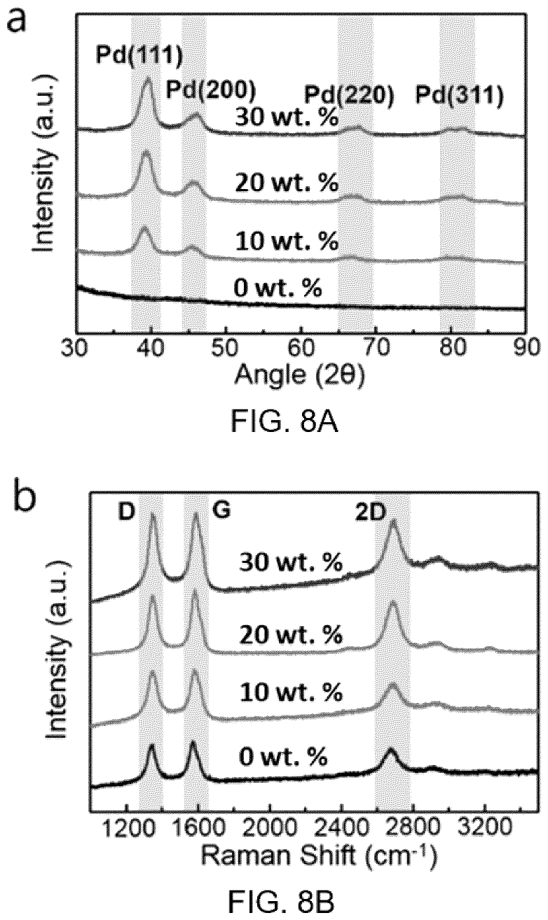

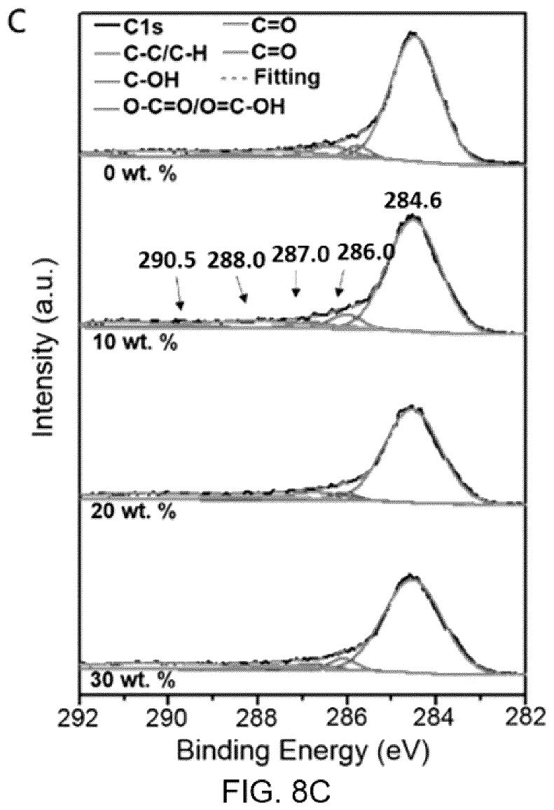

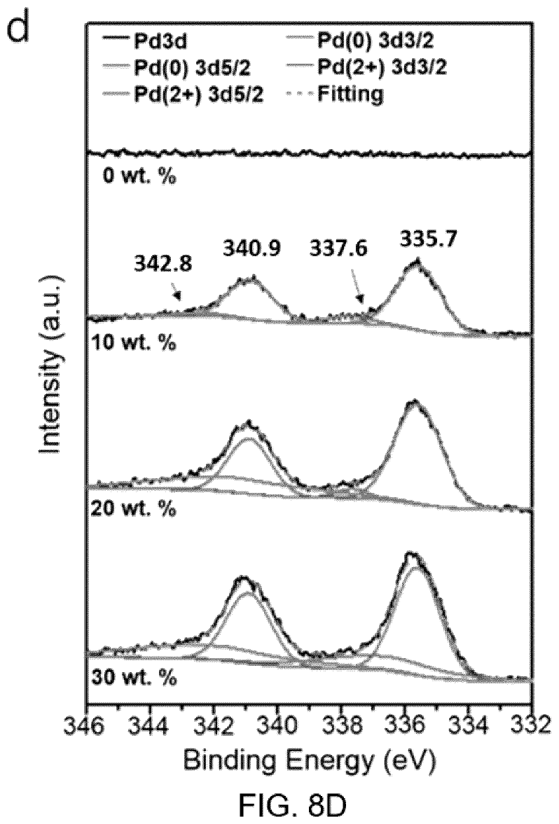

The hybrid structures were characterized using X-ray diffraction (XRD), Raman spectroscopy, and X-ray photoelectron spectroscopy (XPS) to investigate the structural and chemical properties. The crystalline characteristics of PdNPs in the nanoassemblies were analyzed using XRD, and the XRD results are shown in FIG. 8A. All hybrid structures with different PdNP compositions exhibited four identical characteristic peaks at 2θ=39.6°, 45.5°, 66.5°, and 81.5° in the XRD spectra, which corresponded to the (111), (200), (220), and (311) plane reflections of PdNPs, respectively, indicating the atomic arrangements of PdNPs in face-centered cubic (fcc) structures. The sharp peaks of the (111) plane reflection matched well with the prior HR-TEM analysis. The XRD spectra of porous graphene and PdNPs in the nanoassemblies were consistent with the atomic lattice spacing of the PdNPs observed in the TEM data and corroborated the crystalline structures of PdNPs in the hybrid structures.

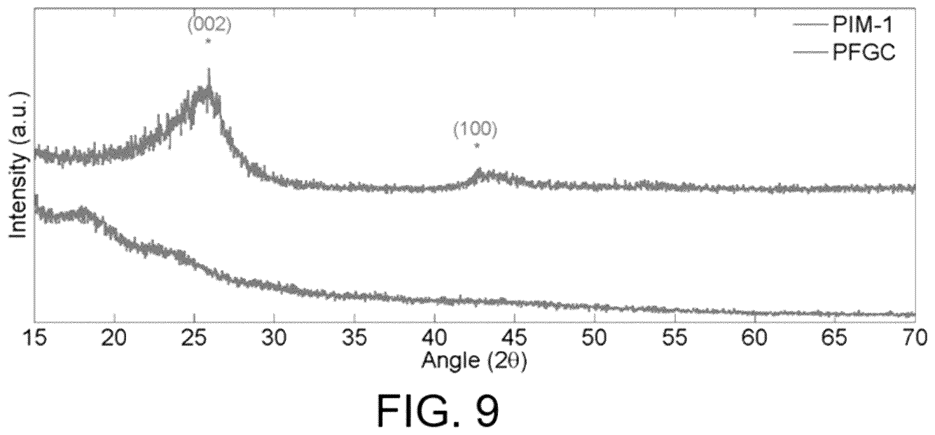

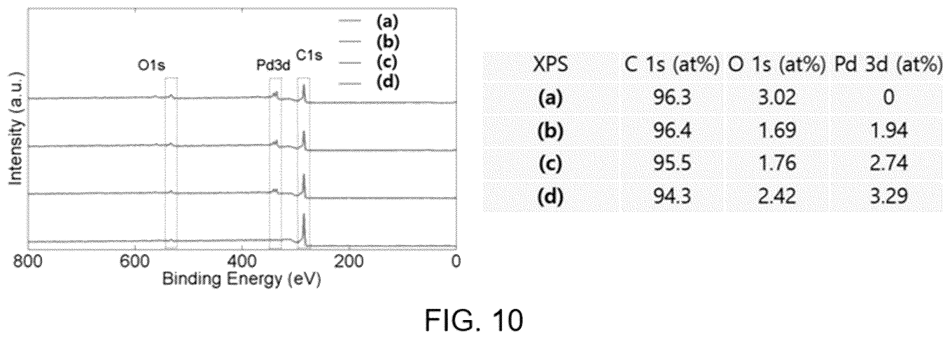

To investigate the crystal structure of porous graphene, XRD was performed on powdered porous graphene, the results of which are in FIG. 9. Characteristic peaks were observed at 2θ values of 42.8° and 25.9°, indicating that the graphene sheets were oriented in the dominant (111) plane and stacked in the (002) plane. The Raman spectra obtained in FIG. 8B shows that all assemblies with different PdNP contents exhibited three distinct peaks at 1350, 1580, and 2690, corresponding to the D, G, and 2D peaks, respectively. The D/G peak intensity ratio of ˜1 and a sharp 2D peak for all hybrid structures with different PdNP concentrations indicated the presence of randomly stacked sp 2-hybridized graphene layers in the porous 3D structure. Moreover, the nanoassemblies with different PdNP compositions showed a consistent D/G intensity ratio, indicating that PdNPs negligibly affected the crystallinity of porous graphene.

To investigate the chemical bonding and compositions of the hybrid structures, XPS analysis was performed, the results of which are shown in FIG. 8C-D, where FIG. C is the spectra for C1s and FIG. D is the spectra for Pd3d. The plain porous graphene without PdNPs showed a characteristic C1s peak at 284.6 eV in XPS data, indicating the presence of predominantly C—C bonds in porous graphene. With the variation of the PdNP composition, the sp²-C content decreased slightly by 2 at. % for 30 wt. % Pd-ligand, with an increase in the 0 content, as seen by the spectra in FIG. 10. This was attributed to the reactions of the liberated volatile compounds, including O in Pd-ligand, with carbon during the transient photon-induced thermal heating. Notably, sp²-C was predominant in the porous graphene with the highest PdNP content. The characteristic peaks of Pd3d, corresponding to the binding energy of Pd, were clearly observed in the spectra (FIG. 8D). The Pd3d peak intensity increased with an increase in the PdNP content. The sharp and intense peaks in the Pd3d spectra indicated the high crystallinity of PdNPs in porous graphene and allowed the loading of up to 3.3 at. % of PdNPs. Porous graphene in the nanoassembly with coherently bonded Pd acted as a p-type transducer. It is notable that laser-induced porous graphene with ebeam-deposited PdNPs and oxide groups containing ˜2 at. % of O has been reported as an n-type transducer.

### 4. Example 3: H₂ Detection

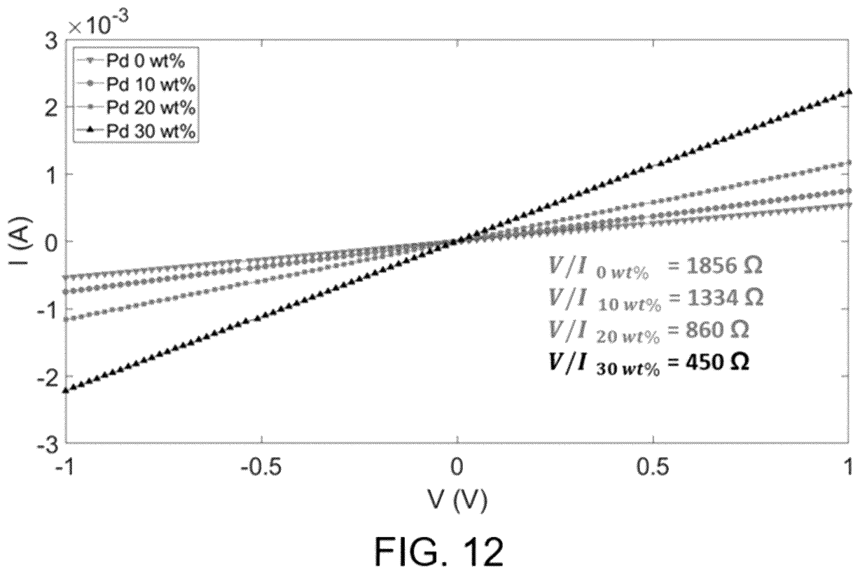

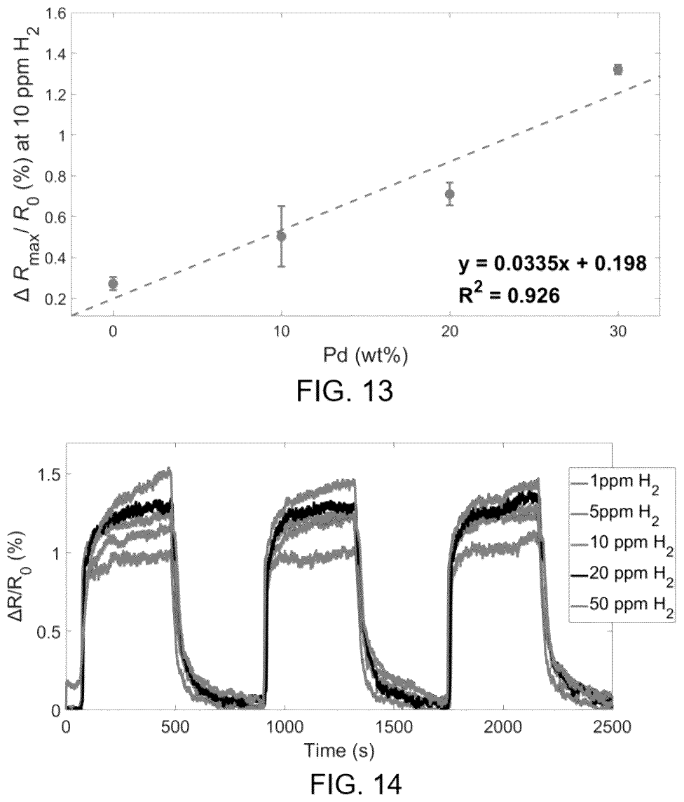

To demonstrate the potential of the nanoassemblies for hydrogen detection, the H₂ detection performance was evaluated using the setup shown in the inset of FIG. 11A. First, the electrical conductivities of the nanohybrids and plain porous graphene were determined. The electrical conductivity of the hybrid increased with an increase in the Pd content. As shown in FIG. 12, the hybrid (30 wt. % Pd-ligand) exhibited a higher electrical conductivity by a factor of approximately four in comparison to plain porous graphene. The enhancement of the conductivity of the nanohybrid was attributable to the higher intrinsic electrical conductivity of Pd metal in comparison to that of porous graphene. The detection limits, response times, and dynamic responses of the nanohybrids with different PdNP concentrations were determined for 10 ppm H₂ (FIG. 11A). The sensitivity, which is defined as the ratio of the maximum resistance change (ΔRₘₐₓ) to initial resistance (Rₒ) (ΔRₘₐₓ/Rₒ (%)) when the hybrid structure is exposed to H₂, increased with increasing the PdNP concentration in porous graphene. The nanohybrids with PdNPs generated from films containing 10, 20, and 30 wt. % Pd-ligand showed the sensitivities of 0.50%, 0.71%, and 1.32%, respectively, whereas the plain porous graphene exhibited no significant change in resistance (FIG. 11A). As shown in FIG. 13, the increase in sensitivity was proportional to the atomic composition of PdNPs in the nanoassembly. The increase in sensitivity with increase in the PdNP content was attributed to the greater number of active sites available with higher concentration of PdNPs to form palladium hydrides (PdHₓ). When the surface of the PdNP is contacted with H₂ molecule, the two hydrogen atoms dissociate, diffusing into the Pd lattice and occupying the interstitial sites to form the complex hybrid, PdHₓ, and reduce the work function of Pd. Thus, the electrons generated during the formation of PdHₓ were transferred to porous graphene because of the lower work function of PdHₓ compared to that of porous graphene. The transferred electrons decreased the number of holes in porous graphene and increased the electrical resistance of porous graphene functioning as the p-type transducer. Thus, both porous graphene and PdNPs played important roles in the sensing mechanism of hydrogen gas detection. Porous graphene afforded high surface area owing to high porosity, as well as loading of high concentration of PdNPs in the 3D porous structures, which significantly increased the number of active sites for hydrogen chemisorption.

The response times (τ) with Pd-ligand contents of 10, 20, and 30 wt. % as well as plain porous graphene were determined for 10 ppm hydrogen at room temperature (RT). The response time is defined as characteristic time required to reach 1/e 36.8%) of ΔRₘₐₓ. The nanohybrids with 10, 20, and 30 wt. % Pd-ligand showed response times of 75.1, 43.1, and 12.1 s. As shown in FIG. 14, the hybrid with a higher Pd content showed a faster response time, and plain porous graphene showed a slower response compared to the nanohybrids. The fast response of the nanohybrid with a high Pd content was attributable to the high PdNP density and relatively large NP size, which afforded a large Pd surface area. The nano-sized PdNPs with a low work function to form PdHₓ allowed faster hydrogen detection in comparison to plain porous graphene.

To further evaluate the hydrogen detection capability of the nanohybrids, the sensitivity and response time of the nanohybrid with 30 wt. % Pd-ligand were determined for 1-50 ppm hydrogen at RT. These results are shown in FIG. 11B. The resistance was decreased by ˜7% at a low H₂ concentration and the decrease is attributed to the low formation of PdHₓ. The hybrid nanostructures showed high sensitivity of 1.2% for the lowest hydrogen concentration of 1 ppm and response time of 7.2 s at 50 ppm. The response time increased by a factor of approximately three for the lowest hydrogen concentration of 1 ppm compared to that for 50 ppm. FIG. 11C shows that repetitive hydrogen detection was demonstrated under cyclic exposure of 1, 5, and 50 ppm hydrogen gas at RT, including, as shown in FIGS. 14, 10 and 20 ppm. The sensors showed fast response and recovery times of 7.2 and 28.1 s with standard deviations of 0.26 and 2.71, respectively, over four cycles at 50 ppm. The ΔR increased upon H₂ injection, and the sensors recovered rapidly as H₂ inflow was stopped and the chamber was vacuumed. The fast and repeatable hydrogen detection was attributed to the reversible hydrogen absorption and desorption reaction with PdNPs and low work function of PdNPs.

### 5. Example 4: Flexible Gas Sensing System

To demonstrate the potential of flexible gas sensing systems based on these nanohybrids, the mechanical reliability of the nanohybrids as well as robustness and durability of the H₂ sensor was evaluated under bending and twisting strains at a hydrogen concentration of 10 ppm. Notably, as shown in FIG. 11D, the sensitivity was consistent upon bending up to 0.22%. FIG. 11E shows that the sensor devices showed reliable detection under 0.22% cyclic bending strain for up to 10,000 cycles, indicating outstanding durability of the nanohybrids and reliable sensing. The mechanical stability of the hybrid structures was also investigated under cyclic twisting with twisting angles between ±90°. The mechanically robust hybrid structures allowed stable detection for up to 10,000 cycles. These results are shown in FIG. 11F. High mechanical flexibility and reliability of 3D porous graphene together with the intrinsic flexibility of PIM-1-based thin film substrates afforded excellent durability under various mechanical strains including bending and twisting. Notably, the TEM images corroborate the presence of strong covalent and metallic carbon-Pd bonds in the hybrid nanostructures, providing high mechanical robustness to the hybrid. These device characterizations under various modes of mechanical strains show the potential of this nanohybrid-based hydrogen sensor as a flexible sensing system.

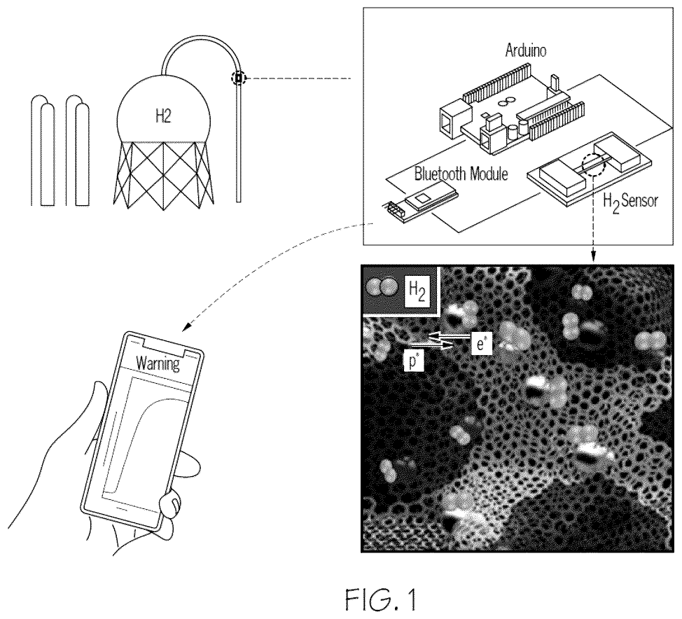

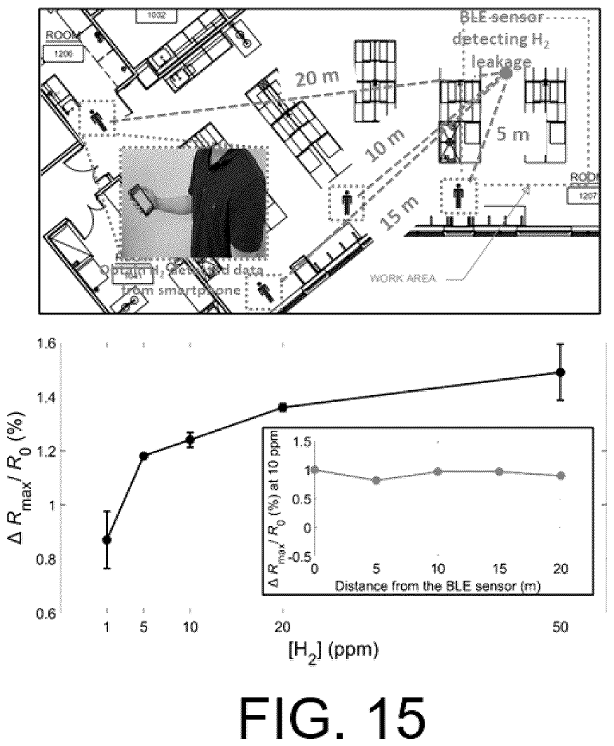

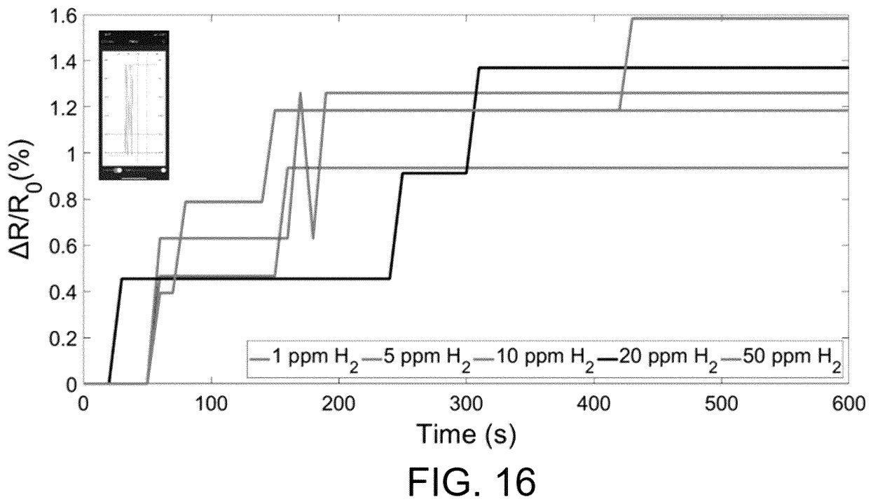

To further develop the flexible sensing systems for wearable H₂ sensors as safety alarm systems in hydrogen generation or storage industries, the nanohybrid-based hydrogen sensor device was integrated with signal processing and wireless communication modules to demonstrate its potential, as shown in FIG. 1. The nanohybrid-based hydrogen sensor device was integrated with signal processing and communicating module that transferred the H₂ sensitivity data for resistance change upon hydrogen exposure to a mobile application. A Bluetooth (BLE) chip (Adafruit Feather 32u4 microcontroller chip) that could transfer the data wirelessly within 20 m distance to the mobile electronic devices was used. Hydrogen detection was performed for various hydrogen concentrations of 1-50 ppm, and the sensing data was received on a smartphone at varying distances of 5-20 m from the sensor to the mobile device. This is demonstrated in FIG. 15. This nanohybrid-based wireless sensing system showed consistent sensitivity for hydrogen concentrations of as low as 1 ppm and varying distances, as shown in FIG. 15 and FIG. 16, in comparison to the previous wired H₂ detection performances

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other methods and systems for carrying out the several purposes of this disclosure. As a result, the claims should not be considered as limited by the description or examples.

What is claimed is:

1. A method of making a hybrid nanomaterial comprising:
a) providing a solution comprising a polymer having intrinsic microporosity and a palladium(II) ligand in an organic solvent, wherein the polymer and the ligand are at least partially soluble in the organic solvent;
b) removing the organic solvent to provide a film comprising the polymer and the ligand; and
c) irradiating the film with an infrared laser to form the hybrid nanomaterial.

2. The method of claim 1, wherein the polymer has the following repeating structure:

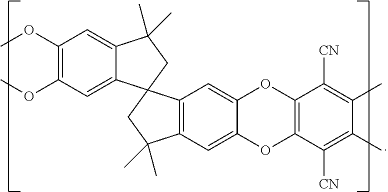

3. The method of claim 1, wherein the palladium(II) ligand is palladium(II) acetate (Pd²⁺(OAc)₂).

4. The method of claim 1, wherein prior to irradiating the film comprises 10-30% by weight of the palladium(II) ligand.

5. The method of claim 1, wherein irradiating is performed with a CO₂ infrared laser having a wavelength (λ) of 10.6 μm.

6. The method of claim 1, wherein the organic solvent is chloroform.

7. A hybrid nanomaterial prepared by the method of claim 1, which comprises porous graphene having palladium nanoparticles dispersed therein.

8. A method of detecting hydrogen gas comprising exposing the hybrid nanomaterial of claim 1 to an environment in which hydrogen gas is to be detected and determining any increase in resistance exhibited by the hybrid nanomaterial while exposed to the environment, thereby indicating the presence or absence of hydrogen gas.

9. The method of claim 8, wherein any increase in resistance is monitored by a computing device in wired or wireless communication with the hybrid nanomaterial.

10. The method of claim 5, wherein the irradiating is performed at 12 Watts, 1,000 laser pulses per inch (PPI), and at a speed of 50 inches per second.

11. A hybrid nanomaterial comprising porous graphene having an x-ray diffraction pattern containing peaks at about 43° and 26° 2θ; and palladium nanoparticles dispersed in the porous graphene, the palladium nanoparticles having an x-ray diffraction pattern containing peaks at about 39°, 45°, 66°, and 81° 2θ.

12. The hybrid nanomaterial of claim 11, wherein the average diameter of the palladium nanoparticles ranges from about 5 nm to about 12 nm as determined by transmission electron microscopy.

13. The hybrid nanomaterial of claim 11, which has a Raman spectrum containing peaks at about 1,350 cm⁻¹, about 1580 cm⁻¹, and about 2690 cm⁻¹.

14. The hybrid nanomaterial of claim 11, wherein the porous graphene has a lattice fringe spacing ranging from about 0.2 nm to about 0.5 nm as determined by transmission electron microscopy.

15. The hybrid nanomaterial of claim 14, wherein the porous graphene has a lattice fringe spacing of about 0.34 nm as determined by transmission electron microscopy.

16. The hybrid nanomaterial of claim 11, wherein the porous graphene has an average pore diameter of 2 nm or less as determined by transmission electron microscopy.

17. The hybrid nanomaterial of claim 11, wherein the palladium nanoparticles have an atomic lattice spacing ranging from about 0.2 to about 0.3 nm as determined by transmission electron microscopy.

18. The hybrid nanomaterial of claim 17, wherein the palladium nanoparticles have an atomic lattice spacing of about 0.23 nm as determined by transmission electron microscopy.

19. The hybrid nanomaterial of claim 11, comprising from about 1% to about 5% palladium nanoparticles by weight of the porous graphene.

20. The hybrid nanomaterial of claim 19, comprising from about 2% to about 4% palladium nanoparticles by weight of the porous graphene.
